(19) United States
(12) Patent Application Publication
(10) Pub. No.: US 2023/0343112 A1
(43) Pub. Date: Oct. 26, 2023

(54) Electronic system for monitoring the state of awareness of an operator in an aircraft, associated method and associated computer program

(71) Applicants: THALES, COURBEVOIE (FR); UNIVERSITE DE BORDEAUX, BORDEAUX (FR); INSTITUT POLYTECHNIQUE DE BORDEAUX, TALENCE (FR); Centre national de la recherche scientifique, PARIS (FR)

(72) Inventors: Bastien BERTHELOT, Merignac (FR); Vincent IBANEZ, Merignac (FR); Loïc BECOUARN, Merignac (FR); Patrick MAZOYER, Merignac (FR); Pierrick LEGRAND, Talence (FR); Eric GRIVEL, Talence (FR)

(21) Appl. No.: 18/304,576

(22) Filed: Apr. 21, 2023

(30) Foreign Application Priority Data
Apr. 22, 2022 (FR) 2203751

(51) Int. Cl. G06V 20/59; B64D 45/00

(52) U.S. Cl. CPC G06V 20/597; B64D 45/00

(57) ABSTRACT

An electronic system for monitoring the state of awareness of an operator in a control station of an aircraft. The monitoring system includes a module for receiving a datum from at least two sensors onboard the aircraft, at least one of the sensors being called a worn sensor being in physical contact with the operator and at least one of the sensors being called an off-set sensor being at a distance from the operator, a processing module configured for extracting from each datum at least one parameter representative of the state of awareness of the operator, a fusion module configured for receiving the representative parameters and implementing a machine learning method for determining, depending on the representative parameters, whether the operator is in a nominal or an altered state of awareness.

### REFERENCE TO RELATED APPLICATION

This application is a U.S. non-provisional application claiming the benefit of French Application No. 22 0351, filed on Apr. 22, 2022, the contents of which are incorporated herein by reference in their entirety.

### TECHNICAL FIELD OF THE INVENTION

The present invention relates to an electronic system for monitoring the state of awareness of an operator in an aircraft.

The invention further relates to a method for controlling the monitoring of the state of awareness of an operator in an aircraft.

The invention further relates to a computer program including software instructions which, when executed by a computer, implement such a method.

### BACKGROUND OF THE INVENTION

The aircraft is typically an airplane, a helicopter, or a drone. The operator is e.g. the pilot of the aircraft, the co-pilot or a remote operator (i.e. drone pilot or radar operator). The control station is in particular arranged in the aircraft or is arranged at a distance from the aircraft in the case e.g. of a drone.

Monitoring the state of awareness of such an operator is essential for the safety of the aircraft in order to detect any a loss of consciousness which could result in the operator's incapacity to perform the tasks expected under operational flight conditions.

Conventionally, the state of awareness of an operator is monitored by the other operators of the aircraft. The co-pilot, e.g., monitors the state of awareness of the pilot, and vice versa. To complement such monitoring, it has been proposed to monitor by means of a sensor arranged in the aircraft.

However, such a method is not entirely satisfactory because the method lacks responsiveness. Indeed, it is necessary to process the data coming from the sensor on a temporal analysis window bringing about a buffering effect in the event of a temporary loss of signal. It is then necessary to wait for the duration of the analysis window, usually a few seconds, before again having valid information on the state of awareness of the pilot. It is thus understood that the responsiveness of such a method is not sufficient, e.g., in case of loss of consciousness of the pilot and where it is important to react quickly, sometimes in less than a second.

Furthermore, such type of method leads to a large number of false positives, in particular when turbulence or vibrations disturb the signal coming from the sensor. It is essential to limit false positives because the countermeasures false positives involve strongly impact the operation of the aircraft: inadvertent issuing of warnings, automatic takeover of the aircraft, external intervention, etc.

Moreover, the known methods are not sufficiently robust with regards to the variability of situations and the limited amount of data. In particular, the monitoring method needs to be robust with regards to the variabilities of the situations encountered. Indeed, the detection of a loss of consciousness has to be robust with regards to the variability of the physiological features of the different operators (age, gender, etc.), and also with regards to the variability of the environment wherein the operator is located (cockpit of an aircraft under turbulence, cockpit where ambient luminosity varies, etc.). Finally, the method has to be robust despite the little operational data available corresponding to the sought for behaviors. In the military field, such a problem is all the more complex because the data collected and useful for the application of interest are not necessarily available since same are often classified and few because of the difficulty of observing critical situations.

There is thus a need for a system for monitoring the state of awareness of an operator, providing better responsiveness while being precise, reliable and robust.

### SUMMARY OF THE INVENTION

To this end, the subject matter of the invention is an electronic system for monitoring the state of awareness of an operator in a control station of an aircraft, the monitoring system including:
a receiver module configured for receiving data from at least two sensors onboard the aircraft, at least one of the sensors called a worn sensor being in physical contact with the operator, and at least one of the sensors called an off-set sensor being at a distance from the operator;
a processing module configured for extracting from each datum, at least one parameter representative of the state of awareness of the operator; and
a fusion module configured for receiving the representative parameters and implementing a machine learning method for determining, according to the representative parameters, whether the operator is in a nominal state of awareness or in an altered state of awareness.

Thereby, the present invention rests on the use of a plurality of sensors of different types, either worn or off-set, and the fusion of the representative parameters obtained by means of the different processing of the data coming from the sensors. The monitoring is thereby more robust with regards to variabilities due to the operator, to the environment and to the flight conditions, while maintaining an increased responsiveness to the detection of a degraded state of awareness of the operator. Indeed, multiplying and diversifying the data collection channels via the different sensors ensures better availability of detection and reduces the number of false positives.

According to other advantageous aspects of the invention, the electronic monitoring system includes one or a plurality of the following features, taken individually or according to all technically possible combinations:
the monitoring system further includes a warning module configured for issuing a warning signal when the fusion module determines that the operator is in an altered state of awareness;
each worn sensor is chosen from the group consisting of:
   a cardiac sensor, in particular an electrocardiograph;
   a pulse oximeter, in particular a photoplethysmography sensor;
   a respiration sensor;
   an accelerometer;
   a scalp electrode, e.g. an electroencephalograph;
   a pressure sensor arranged in an operator's seat;
   a pressure sensor arranged in a control system suitable for being actuated by the operator;
   a sweating sensor for the operator;
   a galvanic skin response sensor;
   an internal temperature sensor for the operator; and
   a near-infrared spectroscopy headband;
at least one of the worn sensors is a pressure sensor configured for measuring at least one pressure applied by the operator to the pressure sensor, the associated parameter suitable for being extracted by the processing module being a duration during which the measured pressure is greater than a predetermined threshold pressure;
at least one of the worn sensors is an accelerometer configured for measuring an acceleration of at least part of the operator, the associated parameter suitable for being extracted by the processing module being a signature resulting from a frequency analysis and/or a temporal analysis of the measured acceleration and chosen from the group consisting of:
   a power carried by a frequency band of the measured acceleration;
   a ratio between the powers of the frequency bands of the measured acceleration;
   the power of the measured acceleration;
   the mean of the measured acceleration;
   the zero-crossing rate of the measured acceleration;
   the regularity of the measured acceleration;
   the complexity of the measured acceleration;
   the entropy of the measured acceleration;
   the parameters of a modeling of the measured acceleration;
   the coefficients resulting from a time frequency analysis of the measured acceleration; and
   the coefficients from a time scale analysis of the measured acceleration;
each off-set sensor is chosen from the group consisting of:
   a camera configured for taking at least one image including at least part of the operator;
   a microphone for picking up at least one sound emitted by the operator, such as the operator's voice or the operator's breath; and
   an infrared sensor for the operator's skin temperature;
at least one of the off-set sensors is a camera configured for taking at least one image including at least a part of the operator, each parameter suitable for being extracted by the processing module being chosen from the group consisting of:
   a movement of the operator;
   a position of the operator;
   an orientation of the head of the operator;
   a direction of the glance of the operator;
   a partial opening of the eyes of the operator;
   a blinking of the eyes of the operator; and
   information on the structure of the image wherein the operator appears;
at least one of the worn sensors is a pressure sensor, at least one of the worn sensors is an accelerometer and at least one of the off-set sensors is a camera; and
the processing module is configured for extracting from each datum, at least one parameter representative of the state of awareness of the operator by implementing, for each datum, an algorithm chosen from the group consisting of:
   an extraction of a predetermined characteristic of the associated datum followed by a machine learning method;
   a deep learning method applied directly to the associated datum; and
   a predetermined modeling applied to the associated datum.

The invention further relates to a method for monitoring the state of awareness of an operator in an aircraft control station, the monitoring method including at least:
reception of a datum from at least two sensors onboard the aircraft, at least one of the sensors called a worn sensor being in physical contact with the operator, and at least one of the sensors called an off-set sensor being at a distance from the operator;
extracting from each datum at least one parameter representative of the state of awareness of the operator; and
reception of the representative parameters and implementation of a machine learning method for determining, according to the representative parameters whether the operator is in a nominal state of awareness or in an altered state of awareness.

The invention further relates to a computer program including software instructions which, when executed by a computer, implement a monitoring method as defined hereinabove.

### BRIEF DESCRIPTION OF THE DRAWINGS

Such features and advantages of the invention will become clearer upon reading the following description, given only as a non-limiting example, and made with reference to the enclosed drawings, wherein:

FIG. 1 is a schematic representation of an aircraft including a monitoring system according to the invention;

FIG. 2 is a schematic representation of control station inside the aircraft shown in FIG. 1; and

FIG. 3 is an organization chart of a monitoring method according to the invention, as used by the electronic system.

### DETAILED DESCRIPTION OF EMBODIMENTS

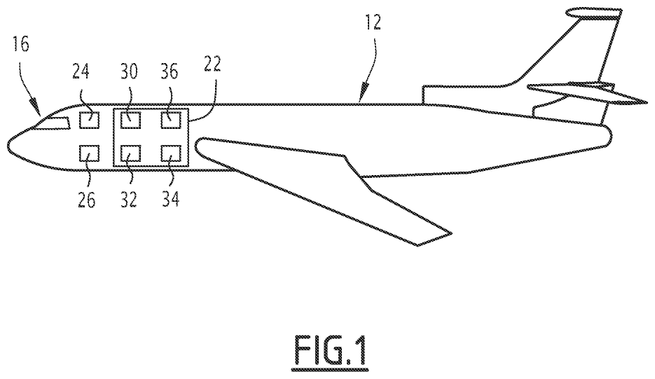

An aircraft 12 is shown in FIG. 1.

Aircraft 12 is typically an airplane, a helicopter, or a drone. In other words, aircraft 12 is a flying machine which may be piloted by an operator 14 via a control station 16. Control station 16 is arranged inside aircraft 12 or at a distance from aircraft 12, in particular in the case of a drone.

Operator 14 is herein a pilot, but the invention applies in a similar manner to any operator of aircraft 12 such as a co-pilot or a radar operator.

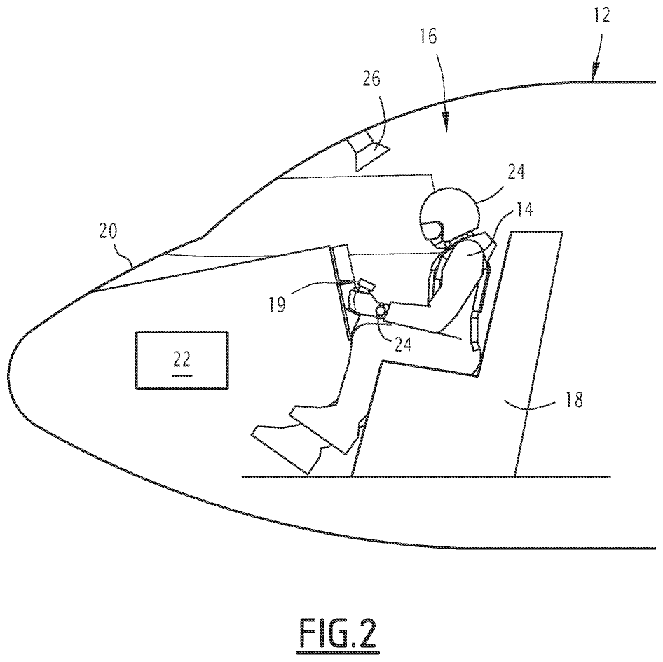

As may be seen in FIG. 2, control station 16 is herein a cockpit of aircraft 12. As may be seen in FIG. 1, control station 16 includes at least one seat 18 for operator 14, a control system 19 suitable for being actuated by operator 14, a windscreen 20 at least partially transparent and separating the inside of the cockpit from the outside environment of aircraft 12, a plurality of sensors, and an electronic monitoring system 22 of the state of awareness of operator 14.

Each sensor is configured for measuring at least one piece of information relating to operator 14 and, in particular, to his/her state of awareness, as will be explained in greater detail thereafter.

Control station 16 includes at least two sensors.

At least one of the sensors is a worn sensor 24 and at least one of the sensors is an off-set sensor 26.

Advantageously, two of the sensors are worn sensors 24 and one of the sensors is an off-set sensor 26.

A worn sensor 24 is a sensor suitable for being in physical contact with operator 14. A person skilled in the art will understand that “in contact” means that sensor 24 touches a part of operator 14, possibly with a garment between the sensor and the skin of operator 14. A worn sensor 24 is thus not necessarily an accessory permanently worn by operator 14 such as a wristwatch, worn sensor 24 being, if appropriate, in contact with operator 14 in a discontinuous manner, such as, e.g., a pressure sensor arranged on a control system 19. Thereby, worn sensor 24 is, e.g., in the form of a watch on the operator's wrist, a helmet on the head of operator 14, or a sensor integrated into control system 19 or into seat 18.

In particular, each worn sensor 24 is chosen from the group consisting of:
a cardiac sensor;
a pulse oximeter;
a respiration sensor;
an accelerometer;
a scalp electrode;
a pressure sensor arranged in a seat 18 of the operator 14;
a pressure sensor arranged in a control system 19 suitable for being actuated by the operator 14;
a sweating sensor for the operator 14;
a galvanic skin response sensor;
an internal temperature sensor for the operator; and
a near-infrared spectroscopy headband.

An off-set sensor 26 is a sensor arranged at a distance from operator 14 when the measurement of off-set sensor 26 is taken, during operational flight conditions. A person skilled in the art will understand that the term “at a distance” means that there is an empty space between sensor 26 and operator 14 during the measurement taken by off-set sensor 26.

In particular, each sensor 26 is chosen from the group consisting of:
a camera configured for taking at least one image including at least part of operator 14;
a microphone for picking up at least one sound emitted by operator 14, such as the operator's voice or the operator's breath; and
an infrared sensor for the skin temperature of operator 14.

In an advantageous embodiment, at least one of worn sensors 24 is a pressure sensor, at least one of worn sensors 24 is an accelerometer, and at least one of off-set sensors 26 is a camera. Thereby, three types of sensors are present in control station 16 which may be used for multiplying and diversifying the data collection channels. Advantageously, a plurality of sensors of each type are present in control station 16, e.g., two cameras, six pressure sensors and four accelerometers.

The electronic monitoring system 22 is configured for monitoring the state of awareness of operator 14. The state of awareness is representative of the ability of operator 14 to become aware of his/her own state and his/her environment in order to react accordingly.

In particular, the state of awareness may be a so-called “nominal” state of awareness, corresponding to the expected state of awareness of operator 14 during a flight of aircraft 12, i.e., an awake and lucid state.

The state of awareness may be a so-called “altered” state of awareness, corresponding to the state of awareness of at least partial loss of awareness of operator 14 with regard to the outside world, such as, e.g., a state of drowsiness, of sleep or of fainting. In such state of awareness, the operator has an altered or non-existent knowledge of his/her environment and cannot react accordingly. Such altered state of awareness is problematic during the flight of aircraft 12 because operator 14 is not able to carry out the tasks he/she has to perform in a reactive and relevant way.

To this end, monitoring system 22 is configured for determining whether operator 14 is in a nominal state of awareness or in an altered state of awareness.

More particularly, monitoring system 22 includes a receiver module 30, a processing module 32, and a fusion module 34.

Advantageously, control station 22 further includes a warning module 36.

Receiver module 30 is configured for receiving a datum from at least two sensors onboard aircraft 12, including at least one worn sensor 24 and at least one off-set sensor 26.

Processing module 32 is configured for extracting from each datum received by receiver module 30, at least one parameter representative of the state of awareness of operator 14.

A parameter representative of the state of awareness is a parameter defined, e.g., by experts in the field and giving information on the state of awareness of the pilot. A low heart rate, eyes closed over a long period of time, constant pressure exerted, a tilted head position, etc., are, e.g., parameters for determining that the operator is in an altered state of awareness.

More particularly, when at least one of worn sensors 24 is a pressure sensor configured for measuring at least one pressure applied by operator 14 to the pressure sensor, the associated parameter suitable for being extracted by processing module 32 is a duration during which the measured pressure is greater than a predetermined threshold. Indeed, such a situation reflects a loss of consciousness of operator 14 who exerts, continuously, a significant pressure on a part of seat 18 or on control system 19.

As a variant or in addition, when at least one of the worn sensors 24 is an accelerometer configured for measuring an acceleration of at least part of operator 14, the associated parameter suitable for being extracted by processing module 32 is a signature resulting from a frequency analysis and/or a temporal analysis of the measured acceleration and chosen from the group consisting of:
a power carried by a frequency band of the measured acceleration;
a ratio between the powers of the frequency bands of the measured acceleration;
the power of the measured acceleration;
the mean of the measured acceleration;
the zero-crossing rate of the measured acceleration;
the regularity of the measured acceleration;
the complexity of the measured acceleration;
the entropy of the measured acceleration;
the parameters of an a priori modeling of the measured acceleration;
the coefficients resulting from a time frequency analysis of the measured acceleration; and
the coefficients resulting from a time scale analysis of the measured acceleration.

Relating to the frequency analysis, processing module 32 is configured for estimating the power carried by frequency bands, or relevant power ratios.

Relating to the temporal analysis, processing module 32 is configured for determining a parameter such as the mean, the power or the zero-crossing rate of the signal, the regularity, the complexity and the entropy of the signal. Different markers can be considered for the above, such as multi-scale entropy or Hurst coefficient which are discussed in detail hereinafter.

The term entropy is used in many fields, from thermodynamics to information theory to statistical mechanics and graph structure.

In information theory, Shannon's entropy has been one of the most popular entropies for over 70 years, but other entropies were developed especially in the 1960s and 1970s with many contributors such as Arimoto and Picard when same were discussing discrete random variables. Some generalizations of Shannon's entropy have been proposed, such as Sharma-Mittal entropy, which depend on two real parameters: α#1 which is called the order, and β#1 which is the degree. When α=β, Tsallis entropy is obtained, whereas when β tends to 1, this leads to Rényi entropy for α>0. In the latter case, when α tends to 1, one finds Shannon's entropy. It should be noted that one ends up with Hartley's entropy (minimum entropy, respectively) when α tends to 0 (+∞, respectively). Finally, the case α=2 corresponds to the collision entropy.

The notion of entropy of a dynamic system with F degrees of freedom was introduced in the late 1950s. The above led to the Kolmogorov-Sinai (KS) entropy, the value of which distinguishes an ordered system from a chaotic system. The above was the starting point of new research work the aim of which was to obtain, in practice, an approximation of KS entropy. Quantities such as the K_2 entropy proposed by Grassberger and Procaccia, the Eckmann-Ruelle entropy, and finally the approximate entropy (ApEn) proposed by Pincus in 1991 [Pincus1991], resulted therefrom.

The “Sample entropy” (SampEn) is an extension of the ApEn; same was proposed by Richman and Moorman twenty years ago. The theoretical expression of SampEn for a white noise was given by Jiang. Although widely used, SampEn is sensitive to short-lived signals. Variants have been developed such as the sample entropy coefficient (COSEn) proposed by Lake and Moorman for solving the problem of atrial fibrillation. The problem of atrial fibrillation corresponds to the SampEn from which terms such as the logarithm of the RR interval (time between two heartbeats) have been subtracted. Chen suggested using the concept of Zadeh fuzzy sets in the classification procedure of ApEn and SampEn, which led to the fuzzy entropy (FuzzyEn). There is also the so-called distribution entropy, the permutation entropy and the hierarchical entropy.

Performing a classification of time series such as the evolution of inter-beat intervals is not necessarily an easy task if only one “scale” of the signal is considered. Consequently, taking into account different scales of the signal can be envisaged. The above leads to so-called multiscale entropies.

Twenty years ago, multiscale entropy (MSE) was proposed by Costa for evaluating complexity of a signal. MSE consists of summing the SampEn of the signal as such and also the SampEn of time series corresponding to what is called a new “scale”, τ, of the signal: such series are deduced as follows: 1/performing, on the original signal, an averaging low-pass with increasingly lower cut-off frequency filtering, when τ increases, and 2/decimating the filtered signal by a factor τ.

In 2009, the refined multiscale entropy was proposed by Valencia. where the finite, impulse response, low-bandpass filter is replaced by an infinite, impulse response low-band pass filter, the cut-off frequency of which is chosen for correctly sub-sampling the signal (i.e., avoiding problems of spectrum overlap when sampling frequency decreases), which was not the case in the standard MSE. However, the proposed filter is not a linear phase filter and introduces phase distortion within the passband. In 2013, the composite multiscale entropy proposed by Wu consisted of combining the τ sequences which may be defined after the decimation step, while the MSE retained only the first. The refined composite multiscale entropy was then implemented.

Taking advantage of entropies alternative to SampEn, other multi-scale entropies have been proposed in recent years. Among same, and without being exhaustive, the multiscale permutation entropy, the multiscale fuzzy sample entropy (MFE), the multiscale composite fuzzy entropy, composite and refined, the refined multiscale composite permutation entropy, and the generalized multiscale entropy, may be cited.

The analysis of the Hurst exponent of a signal will now be explained. Such analysis may be done by different methods. The prior art comes down to two main families: the estimators based on frequency analysis of the signal, and the estimators based on temporal analysis of the signal. There are also methods based on evolutionary algorithms.

In order to estimate the Hurst coefficient of a mono-fractal process, the method called “Fluctuation Analysis” (FA) may be implemented. The principle is as follows: after integration of the signal leading to a new sequence yᵢₙₜ, the following quantity is calculated for different values of l: F(N)={square root over (<(yᵢₙₜ(i+N)−yᵢₙₜ(i))²>)}, where <.> is the time average. Since F(N)∝N^{H}, where ∝ represents a proportionality relationship, log(F(N)) is then represented as a function of log(N) in order to estimate the value of H: the latter is equal to the value of the slope of the regression line.

It is possible to use DFA (Detrended Fluctuation Analysis) or DMA (Detrended Moving Average Analysis) and the variants thereof, for estimating H. Same are based on the same principle: the analysis of fluctuations around a trend of the centered and integrated signal. The analysis is then conducted on a process called “the residue”, defined as the difference between the integrated version of the signal and the trend.

As a variant or in addition, at least one of the off-set sensors is a camera configured for taking at least one image including at least a part of operator 14, each parameter suitable for being extracted by processing module 32 being chosen from the group consisting of:
a movement of operator 14;
a position of operator 14;
an orientation of the head of operator 14;
a direction of the glance of operator 14;
a partial opening of the eyes of operator 14;
a blink of the eyes of operator 14; and
information on the structure of the image wherein operator 14 appears.

Information on the structure of the image wherein operator 14 appears is, e.g., an analysis of the distribution of the colors of the pixels of the image. A nominal state is associated with a nominal distribution with, e.g., a blue scale associated with the sky, a gray scale associated with the aircraft cabin, a beige scale associated with operator 14, etc. A different distribution from such nominal distribution may be a sign of an altered state of awareness of operator 14. E.g., an all-gray image may be a sign of an unconscious operator obstructing the camera with his/her body. Such a method thus does not require an operation of detecting shapes present in the image, and has high availability and speed.

Advantageously, processing module 32 is configured for extracting from each datum at least one parameter representative of the state of awareness of operator 14 by performing an extraction of a predetermined characteristic of the associated datum followed by a machine learning method.

As an example, the characteristic is the position of the head of operator 14, extracted from a video taken by a camera. A machine learning method is then implemented for processing position of the head over time and for inferring therefrom a parameter representative of the state of awareness of the operator 14.

A machine learning method is used for obtaining a model apt to solve tasks without being explicitly programmed for each of the tasks. Machine learning includes two phases. The first phase consists of defining a model from data present in a learning database, also called observations. The definition of the model consists herein, in particular, in training the model to recognize a loss of consciousness. The so-called learning phase is generally carried out prior to the practical use of the model. The second phase corresponds to the use of the model: the model being defined, new data may then be submitted to the model in order to determine the state of awareness of operator 14.

In a variant, processing module 32 is configured for extracting from each datum, at least one parameter representative of the state of awareness of operator 14 by implementing a deep learning method applied directly to the associated datum.

A deep learning method is a technique based on the model of neural networks: tens or even hundreds of layers of neurons are stacked for bringing greater complexity to the model. In particular, a neural network generally consists of a succession of layers, each of which takes the inputs thereof from the outputs of the preceding layer. Each layer consists of a plurality of neurons, taking the inputs thereof from the neurons of the preceding layer. Each synapse between neurons is associated with a synaptic weight, so that the inputs received by a neuron are multiplied by the weight and then added by the neuron. The neural network is optimized by adjusting the different synaptic weights during training, according to the data in the learning database. The neural network thereby optimized is then the model. A new set of data may then be given at the input to the neural network, which then supplies the result of the task for which the neural network has been trained.

In a variant, processing module 32 is configured for extracting from each datum, at least one parameter representative of the state of awareness of operator 14 by implementing a predetermined modeling applied to the associated datum.

The predetermined modeling is, e.g., a physical model including a set of rules predetermined by an expert.

Fusion module 34 is further configured for applying a model coming from a machine learning method, for determining, according to the representative parameters, whether operator 14 is in a nominal state of awareness or in an altered state of awareness.

The machine learning method used by fusion module 34 is different, if appropriate, from the method used by processing module 32.

The machine learning method is trained upstream of the operational phases of flight by studying which parameters are significant and relevant for characterizing the state of awareness of operator 14, e.g., with the help of experts. Experimental data or feedback from past flights can be used.

Warning module 36 is configured for issuing a warning signal when fusion module 34 determines that the operator is in an altered state of awareness.

The warning signal is, e.g., an audible signal emitted in control station 16 for returning operator 14 to a nominal state of awareness.

In a variant or in addition, the warning signal is, e.g., a signal sent to a control system of aircraft 12 in order to switch to automatic mode, and so that the tasks to be performed by operator 14 are carried out autonomously without the intervention of operator 14. In particular, when operator 14 is a pilot, aircraft 12 switches to autopilot.

In a variant or in addition, the warning signal is, e.g., a communication signal to a control system external to aircraft 12 such as a control tower.

In the example shown in FIG. 1, electronic monitoring system 22 includes an information processing unit consisting, e.g., of a memory and of a processor associated with the memory. Receiver module 30, processing module 32, fusion module 34, and warning module 36 are each implemented in the form of a software program, or a software brick, which may be run by the processor. The memory is then apt to store a receiver software, a processing software, a fusion software and, as an optional addition, a warning software. The processor is then apt to run each of the software programs.

In a variant (not shown), receiver module 30, processing module 32, fusion module 34, and, as an optional addition, warning module 36 are each produced in the form of a programmable logic component, such as an FPGA (Field Programmable Gate Array), or further in the form of a dedicated integrated circuit, such as an ASIC (Application Specific Integrated Circuit).

When electronic monitoring system 22 is produced in the form of one or a plurality of software programs, i.e., in the form of a computer program, same is further apt for being recorded on a computer-readable medium (not shown). The computer-readable medium is, e.g., a medium apt to store the electronic instructions and to be coupled to a bus of a computer system. As an example, the readable medium is an optical disk, a magneto-optical disk, a ROM memory, a RAM memory, any type of non-volatile memory (e.g., EPROM, EEPROM, FLASH, NVRAM), a magnetic card or an optical card. A computer program containing software instructions is then stored on the readable medium.

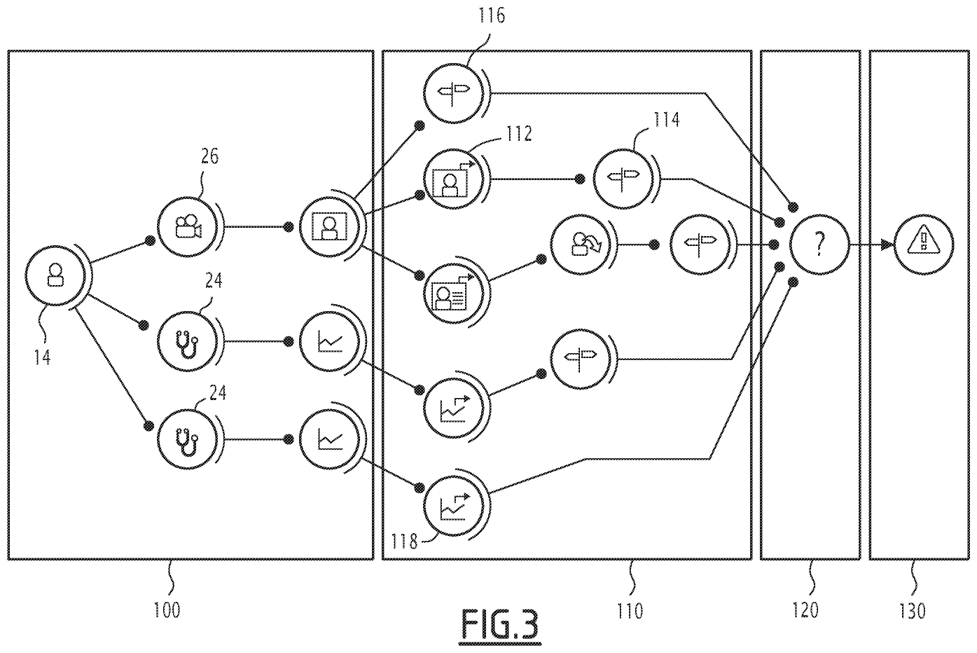

The operation of electronic monitoring system 22 according to the invention will now be explained using FIG. 3 which shows a flow chart of a method according to the invention, for monitoring the state of awareness of operator 14 in control station 16 of aircraft 12.

Initially, aircraft 12 is in an operational flight situation, flying e.g. to an airport.

At least one operator 14 is present in control station 16 of aircraft 12. An operator 14 is, e.g., a pilot, as shown herein.

Control station 16 includes at least two sensors, at least one of which is a sensor 24 worn by operator 14, and at least one of the sensors is an off-set sensor 26 at a distance from operator 14.

The method includes an initial operation 100 of reception by receiver module 30 of a datum from at least one worn sensor 24 and at least one datum from off-set sensor 26.

As an example, worn sensor 24 is a cardiac sensor and the data measured are the heartbeats over time. Off-set sensor 26 is, e.g., a camera placed facing operator 14 in control station 16 and the associated datum is a succession of images over time of the chest of operator 14.

The data being raw, they are advantageously processed. In particular, the data are normalized and centered. Preprocessing further includes checking of data sampling, presence of artifacts or of sensor noise. Preprocessing may also include filtering for removing a trend of the signals, i.e., low frequency elements which are not relevant for monitoring.

The method then includes an operation 110 of extracting, by means of processing module 32 from each datum, at least one parameter representative of the state of awareness of operator 14.

More particularly, processing module 32 extracts from each datum, at least one parameter representative of the state of awareness of operator 14, by carrying out:
extraction 112 of a predetermined characteristic of the associated datum followed by implementation 114 of a machine learning method;
implementation 116 of a deep learning method applied directly to the associated datum; or
predetermined modeling 118 applied to the associated datum.

Still in the same example, processing module 32 extracts, from the datum of worn sensor 24, a heart rate compared to a predetermined rest heart rate. Processing module 32 extracts, from the data of off-set sensor 26, e.g., a frequency and a duration of blinking of the eyes of operator 14 and/or, e.g., a position of the head of operator 14.

The method then includes an operation 120 of receiving, by fusion module 34, the representative parameters and implements a machine learning method, for determining, depending on the representative parameters, whether operator 14 is in a nominal state of awareness or in an altered state of awareness.

Still in the same example, fusion module 34 receives the heart rate and the frequency and a duration of blinking of the eyes and deduces therefrom, the state of awareness of operator 14. The merging of the two parameters gives a better estimation. Indeed, the heart rate of operator 14 may be quite high and not be associated with a loss of consciousness, but the long duration of the blinking of the eyes reflects an altered state of awareness of operator 14. Conversely, the eyes of the pilot may remain open normally, yet a low heart rate may indicate a drowsiness of the pilot.

Advantageously, the method includes an operation 130 of issuing a warning signal when detection module 34 determines that the operator is in an altered state of awareness.

In this way, it may be understood that the present invention has a certain number of advantages.

Indeed, the use of a plurality of sensors of different types and the merging of representative parameters may be used for producing a system for monitoring the state of awareness of an operator providing a better responsiveness while being precise, reliable and robust.

In particular, the monitoring system according to the invention is more robust with regards to variabilities due to the operator, to the environment and to the flight conditions, while maintaining increased responsiveness to the detection of a degraded state of awareness of the operator by multiplication and diversification of the data collected via the different sensors.

Finally, the invention may be used for improving the availability of detection of loss of consciousness, and for reducing the number of false positives.

1. An electronic system for monitoring the state of awareness of an operator in a control station of an aircraft, the monitoring system comprising:
a receiver module configured for receiving a datum from at least two sensors on board the aircraft, at least one of the sensors called a worn sensor being in physical contact with the operator, and at least one of the sensors called an off-set sensor being at a distance from the operator;
a processing module configured for extracting from each datum, at least one parameter representative of the state of awareness of the operator; and
a fusion module configured for receiving the representative parameters and implementing a machine learning method for determining, depending on the representative parameters, whether the operator is in a nominal state of awareness or in an altered state of awareness.

2. The monitoring system according to claim 1, further comprising a warning module configured for issuing a warning signal when said fusion module determines that the operator is in an altered state of awareness.

3. The monitoring system according to claim 1, wherein each worn sensor is chosen from the group consisting of:
a cardiac sensor;
a pulse oximeter;
a respiration sensor;
an accelerometer;
a scalp electrode;
a pressure sensor arranged in a seat of the operator;
a pressure sensor arranged in a control system suitable for being actuated by the operator;
a sweating sensor for the operator;
a galvanic skin response sensor;
an internal temperature sensor for the operator; and
a near-infrared spectroscopy headband.

4. The monitoring system according to claim 3, wherein at least one of the worn sensors is a cardiac sensor comprising an electrocardiograph.

5. The monitoring system according to claim 3, wherein at least one of the worn sensors is a pulse oximeter comprising a photoplethysmography sensor.

6. The monitoring system according to claim 3, wherein at least one of the worn sensors is a scalp electrode comprising an electroencephalograph.

7. The monitoring system according to claim 3, wherein at least one of the worn sensors is a pressure sensor configured for measuring at least one pressure applied by the operator to the pressure sensor, the associated parameter suitable for being extracted by said processing module being a duration during which the measured pressure is greater than a predetermined threshold.

8. The monitoring system according to claim 3, wherein at least one of the worn sensors is an accelerometer configured for measuring an acceleration of at least part of the operator, the associated parameter suitable for being extracted by said processing module being a signature resulting from a frequency analysis and/or a temporal analysis of the measured acceleration and chosen from the group consisting of:
a power carried by a frequency band of the measured acceleration;
a ratio between the powers of the frequency bands of the measured acceleration;
a power of the measured acceleration;
a mean of the measured acceleration;
a zero-crossing rate of the measured acceleration;
a regularity of the measured acceleration;
a complexity of the measured acceleration;
an entropy of the measured acceleration;
parameters of a modeling of the measured acceleration;
coefficients resulting from a time frequency analysis of the measured acceleration; and
coefficients resulting from a time scale analysis of the measured acceleration.

9. The monitoring system according to claim 1, wherein each off-set sensor is chosen from the group consisting of:
a camera configured for taking at least one image including at least part of the operator;
a microphone for picking up at least one sound emitted by the operator; and
an infrared sensor for the skin temperature of the operator.

10. The monitoring system according to claim 9, wherein the sound emitted by the operator is the operator's voice or the operator's respiration.

11. The monitoring system according to claim 9, wherein at least one of the off-set sensors is a camera configured for taking at least one image comprising at least a part of the operator, each parameter suitable for being extracted by said processing module being chosen from the group consisting of:
a movement of the operator;
a position of the operator;
an orientation of the head of the operator;
a direction of the glance of the operator;
a partial opening of the eyes of the operator;
a blink of the eyes of the operator; and
information on the structure of the image wherein the operator appears.

12. The monitoring system according to claim 1, wherein at least one of the worn sensors is a pressure sensor, at least one of the worn sensors is an accelerometer, and at least one of the off-set sensors is a camera.

13. The monitoring system according to claim 1, wherein said processing module is configured for extracting from each datum at least one parameter representative of the state of awareness of the operator by implementing, for each datum, an algorithm chosen from the group consisting of:
an extraction of a predetermined characteristic of the associated datum followed by a machine learning method;
a deep learning method applied directly to the associated datum; and
a predetermined modeling applied to the associated datum.

14. A method for monitoring the state of awareness of an operator in a control station of an aircraft, the monitoring method comprising at least the following steps:
reception of data from at least two sensors onboard the aircraft, at least one of the sensors being called a worn sensor being in physical contact with the operator, and at least one of the sensors being called an off-set sensor being at a distance from the operator;
extraction from each datum, at least one parameter representative of the state of awareness of the operator; and
reception of the representative parameters and implementation of a machine learning method for determining, depending on the representative parameters, whether the operator is in a nominal state of awareness or in an altered state of awareness.

15. A non-transitory computer program including software instructions which, when executed by a computer, cause the computer to perform a method according to claim 14.
